(19) United States
(12) Patent Application Publication
(10) Pub. No.: US 2022/0104772 A1
(43) Pub. Date: Apr. 7, 2022

(54) Smart Phone Cover with Sensors and Method

(71) Applicants: Chad L. Sayers, South Jordan, UT (US); Eric Preston Thomas, North Ogdan, UT (US); Wenyu Liu, Grayslake, IL (US); Baishun Tan, Shenzhen (CH); Rong Zeng, Shenzhen (CH); Hongwei SHI, Shenzhen (CH); Xiaorang Li, Shenzhen (CH)

(72) Inventors: Chad L. Sayers, South Jordan, UT (US); Eric Preston Thomas, North Ogdan, UT (US); Wenyu Liu, Grayslake, IL (US); Baishun Tan, Shenzhen (CH); Rong Zeng, Shenzhen (CH); Hongwei SHI, Shenzhen (CH); Xiaorang Li, Shenzhen (CH)

(21) Appl. No.: 17/493,821

(22) Filed: Oct. 4, 2021

Related U.S. Application Data
(60) Provisional application No. 63/086,950, filed on Oct. 2, 2020.

(51) Int. Cl. A61B 5/00; A61B 5/0205; A61B 5/021; A61B 5/024; A61B 5/08; A61B 5/1455; A61B 5/332; A61B 5/1495; H04M 1/02

(52) U.S. Cl. CPC A61B 5/6887; A61B 5/02055; A61B 5/021; A61B 5/02438; A61B 5/0816; A61B 2562/0271; A61B 5/332; A61B 5/1495; A61B 5/742; A61B 5/002; H04M 1/0202; A61B 5/14552

(57) ABSTRACT

A smartphone cover with a sensor to determine body wellness characteristics of a subject and method is disclosed. A cover having sensors for determining body characteristics is coupled with a mobile communications device. An application on the mobile communications device that signals via a communications channel test criteria to an application executing by a processor in the cover. Sensors embedded in the cover are engaged with the subject to detect the subject's body characteristics to determine medical conditions of the subject. Conditions are indicated on the mobile communications device.

### PRIOR APPLICATION

This application claims the benefit of U.S. provisional application No. 63/086,950 filed on Oct. 2, 2020, the contents of which are hereby incorporated by reference.

### TECHNICAL FIELD

These claimed embodiments relate to a smartphone cover with sensors and more particularly to a smartphone cover coupled to a mobile communications device and method used to test body wellness characteristics.

### BACKGROUND OF THE INVENTION

A Smartphone Cover with sensors and method is disclosed.

In the medical field, sensing devices have been used for determining characteristics of a patient. These sensing devices may be bulky and include a large number of components making it difficult for a user to transport the sensing device on a daily basis.

Body sensor's have been placed in watches and other devices that may be attached to a user. These body sensors may become lost, misplaced or forgotten when used on a daily basis. These sensors may be limited in functionality as their size must be extremely small. Such limited functionality prevents detection of certain pathogens/health conditions may only be detected with a large number of sensors.

Finally, covers have been placed on mobile devices to protect the mobile device. But the covers have been made rugged to ensure sufficient mobile device protection. Such covers have not included sensors to detect body functionality as they may break when dropped.

### SUMMARY OF THE INVENTION

In one implementation, a smartphone cover with sensors is disclosed for determining with a mobile communications device body characteristic of a subject. The method includes engaging a cover having sensors for detecting body characteristics with the mobile communications device. The cover at least partially encloses the mobile communications device. An application is executed on the mobile communications device that signal test criteria via a communications channel to a program executing on the cover. Sensors embedded in the cover engage with the subject to detect the subjects body characteristics. In response to the subject engaging with the sensors, the cover tests the body characteristics of the subject. A signal is from the cover to the mobile communications device via the communications channel indicating at least partial results of the test, and the results of the test are displayed on the mobile communications device.

In another implementation, a mobile communications device cover includes an outside wall and a bottom panel for engaging with the mobile communications device to at least partially enclose the mobile communications device. Sensors are coupled with the bottom panel for determining body characteristics. A processor is coupled with the cover to communicate with the mobile communications device via a communications channel, to receive one or more signals from the mobile communications device to initiate one or more test criteria, to respond to the one or more signals to execute a test application that engages sensors embedded in the cover to sense the subjects body characteristics, and to send a signal with the processor from the cover to the mobile communications device via the communications channel. The signal indicates the sensed body characteristics, such that the one or more tests results are displayed on the mobile communications device in response to reception of the signal indicating the sensed body characteristics.

In a further implementation, a method of detecting an illness of a user with a mobile communications device cover having a processor coupled with a plurality of sensors includes engaging the mobile phone cover with the mobile communications device to attach to and at least partially enclose the mobile communications device. An application is executed with a processor on the mobile communications device that signals via a communications channel one or more test criteria to an application executing by a processor in the cover. Sensors embedded in the cover engage with the subject to detect the subject's body characteristics, the sensors including at least two of an infrared thermo Sensor, a Pulse oximetry (SpO2) sensor, a heart rate sensor, an Eco-Cardiogram (EKG) Sensor, a Blood Pressure Sensor, a temperature sensor, a breathing rate sensor, and a respiratory level sensor. In response to the subject engaging with the sensors and the received one or more test criteria, sensors in the cover detect one or more body characteristics of the subject. The body characteristics of the subject include at least one of temperature, heart rate, EKG, pulse oximetry, blood pressure, breathing rate and respiratory level. The processor in the cover sends a signal to the mobile communications device via the communications channel that indicates the sensed body characteristics. At least one of a health condition or a pathogen of the subject is determined by the mobile communications device processor based on the indicated sensed body characteristics. At least one of the health condition or the pathogen of the subject is displayed on the mobile communications device in response to reception of the signal indicating the sensed body characteristics.

### BRIEF DESCRIPTION OF THE DRAWINGS

The detailed description is described with reference to the accompanying figures. In the figures, the left-most digit(s) of a reference number identifies the figure in which the reference number first appears. The use of the same reference number in different figures indicates similar or identical items.

FIG. 1A is a perspective view and FIG. 1B is a rear view of the smartphone cover in accordance with the invention;

FIG. 2 is a simplified schematic diagram of the smartphone cover;

FIG. 3 is a simplified schematic diagram of the smartphone cover coupled with a smart mobile communications device;

FIG. 4 is a flow chart of the process for testing body characteristics with the smartphone cover; and

FIG. 5 is a flow chart of the process for testing body characteristics with the smart mobile communications device.

### DETAILED DESCRIPTION

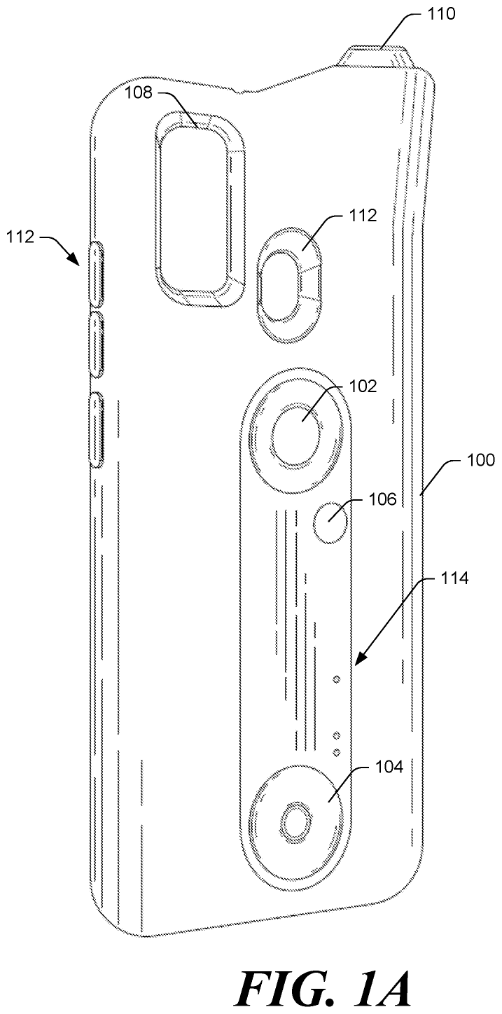

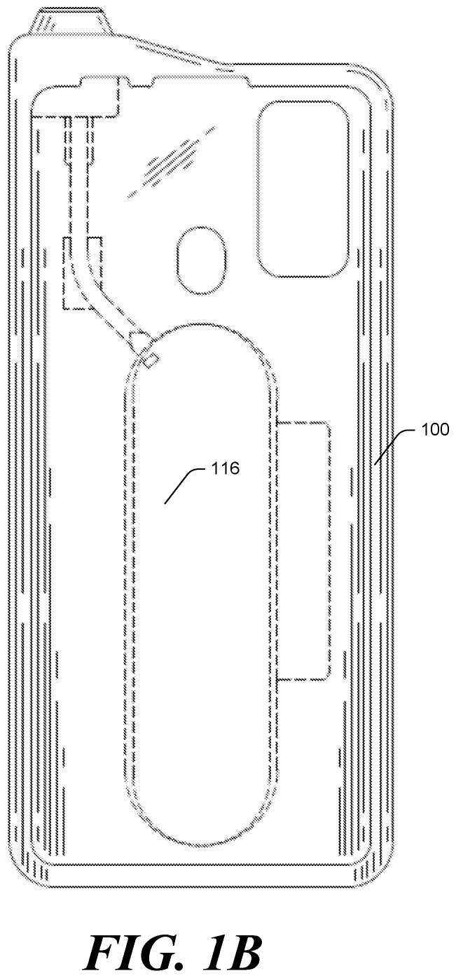

Referring to FIGS. 1A-1B, there is shown a smartphone cover 100 for at least partially enclosing a smart mobile communications device (also referred to as a smartphone and is not shown). The cover is molded to cover the back and sides of any smartphone while allowing the display of the smartphone to be visible and unobstructed at least partially. The mobile communications device may be any communications device having a display and a communications channel, including but not limited to, a smartphone, an iPhone, an Android based phone, an iPad, a PDA, a personal communications device, and a personal assistant device.

The smartphone cover 100 includes medical module pads 102 and 104, inferred thermometer/power on button 106, camera aperture 108, fingerprint reader aperture 112, indicator LED's 114, inferred thermometer probe 110 and apertures for phone keys 112. Pads 102 and 104 may be used to detect a user's IRT (core body temperature), SpO₂ (Blood Oxygen level) heart rate, ECG (Electro-cardiogram) and Blood Pressure. Inferred thermometer probe 110 may detect a user's core body temperature.

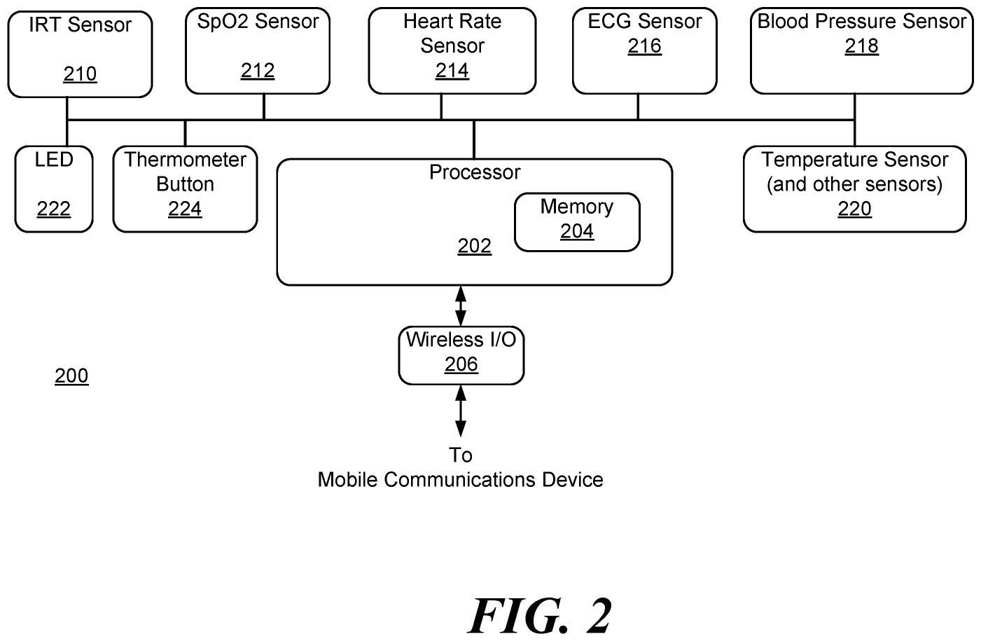

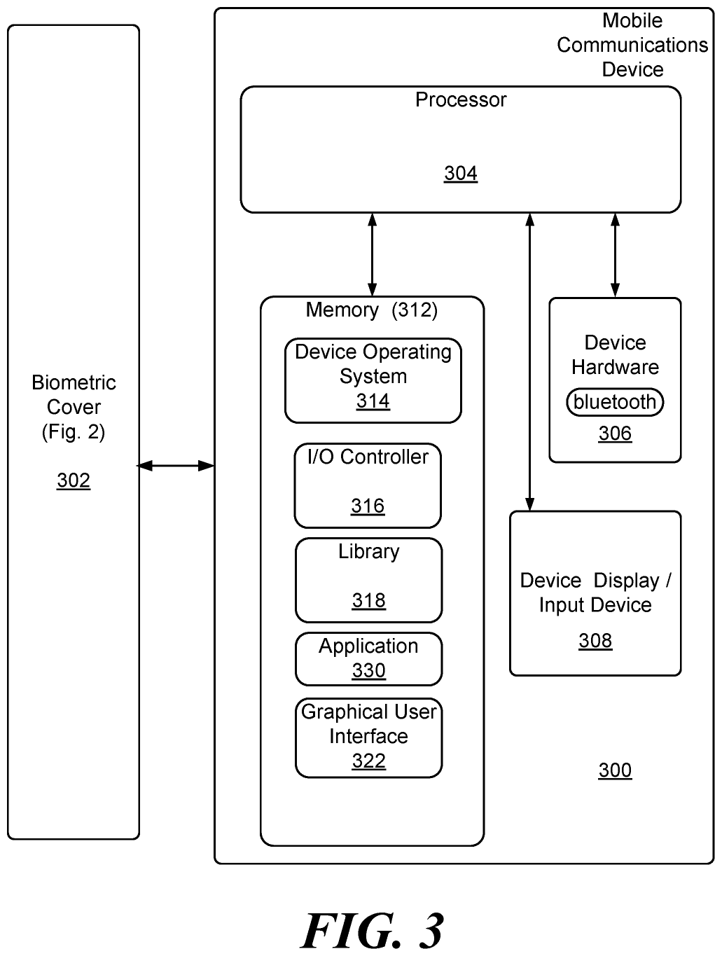

Referring to FIG. 1B, a circuit 116 (See FIG. 2) is integrally coupled to cover 100. Circuit 116 includes a battery (not shown) and processor. Circuit 116 may be electrically coupled with medical module pads 102 and 104, inferred thermometer button 106, indicator LED's 114, inferred thermometer probe 110.

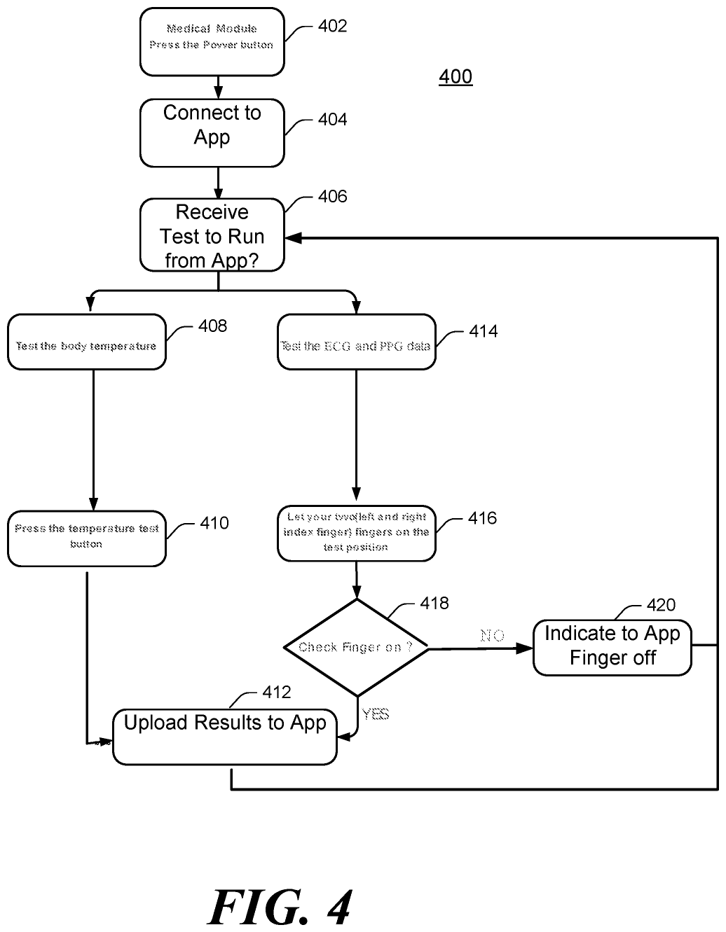

Referring to FIG. 2, there is shown a simplified schematic diagram of a circuit 200 (Circuit 116 in FIG. 1) for monitoring a user's health/medical characteristics. Circuit 200 includes a processor 202 having a memory 204 for storing program instructions other code for executing the processes shown or describe in connection with FIG. 4. Processor 202 may be coupled with a wireless (or wireline) Input/Output (I/O) device such as a Bluetooth communications device 206. Processor 202 may be coupled with IRT Sensor 210, Pulse oximetry (SpO2) sensor 212, heart rate sensor 214, Eco-Cardiogram (EKG) Sensor 216, Blood Pressure Sensor 218, and temperature sensor 220, blood sugar sensor (not shown), and breathing rate sensor or respiratory rate (breathing rate) sensor (not shown). Inferred thermo (IRT) Sensor 210, pulse oximeter (SpO2) sensor 212, heart rate sensor 214, EKG Sensor 216, Blood Pressure Sensor 218 may be separate devices within cover or may be implemented as single sensor (such as medical module pads 102 and 104) in combination with processor 202 running software or firmware instructions stored in memory 204. Processor 202 may be coupled with light emitting diodes 222 and thermometer and/or medical module power on button 224 (Button 106 of FIG. 1).

Referring to FIG. 3, there are illustrated selected modules in smartphone 300 (also referred to herein as a mobile communications device). Smartphone 300 communicates with Cover 302 (Also referred to as a biometric cover). Smartphone 300 includes a processing device 304, memory 312, and display/input device 308. Processing device 304 may include a microprocessor, microcontroller or any such device for accessing memory 312 and display/input device 308. Processing device 304 has processing capabilities and memory suitable to store and execute computer-executable instructions. In one example, processing device 304 includes one or more processors.

Processing device 304 executes instructions stored in memory 312, and in response thereto, processes signals from and to display/input device 308 and device hardware 306 which may include a clock/timer. Device hardware 306 may include input device, network I/o device (not shown) that includes network and communication circuitry (e.g. Bluetooth circuitry, near field communications, and wifi, etc.) for communicating with a communications network and output device 329 for communicating with a wireless I/O device 206 in cover 200 (FIG. 2). Input device 308 receives inputs from a user of the personal computing device and may include a keyboard, mouse, track pad, microphone, audio input device, video input device, or touch screen display. Display device 308 may include an LED, LCD, CRT, or any type of display screen.

Memory 312 (and memory 204) may include a non-transitory volatile and nonvolatile memory, removable and non-removable media implemented in any method or technology for storage of information, such as computer-readable instructions, data structures, program modules or other data. Such memory includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, RAID storage systems, or any other medium (including a non-transitory computer readable storage medium) which can be used to store the desired information, and which can be accessed by a computer system.

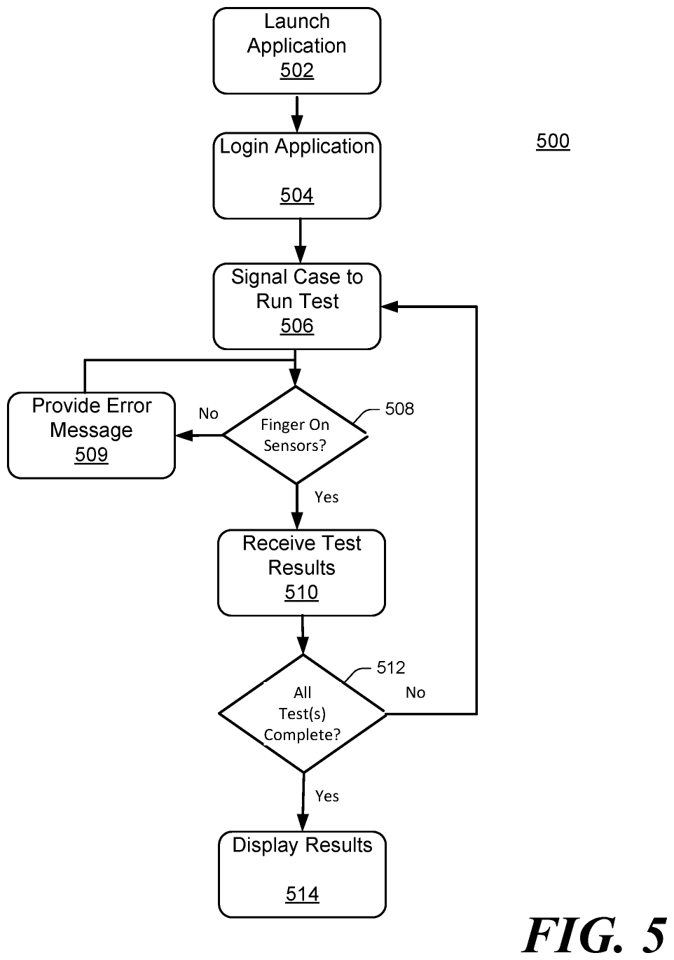

Modules stored in memory 312 of the computing device 208 may include an operating system 314, an I/O controller 316, a library 318, an application 330 and a graphical user interface 322. Operating system 314 may be used by application 330 to operate Display 308. Library 316 may include preconfigured parameters (or set by the user before or after initial operation) such as computing device operating parameters and configurations. Application 330 may include a body characteristic testing programs and other code for executing the processes shown or describe in connection with FIG. 5.

Illustrated in FIG. 4 and FIG. 5, there is shown a process 400 and 500 respectively for body characteristics using processor 202 and 302. The exemplary processes in FIGS. 4 and 5 are illustrated as a collection of blocks in a logical flow diagram, which represents a sequence of operations that can be implemented in hardware, software, and a combination thereof. In the context of software, the blocks represent computer-executable instructions that, when executed by one or more processors, perform the recited operations. Generally, computer-executable instructions include routines, programs, objects, components, data structures, and the like that perform functions or implement particular abstract data types. The order in which the operations are described is not intended to be construed as a limitation, and any number of the described blocks can be combined in any order and/or in parallel to implement the process. For discussion purposes, the processes are described with reference to FIGS. 4 and 5, although it may be implemented in other system architectures.

Referring to FIGS. 4-5, processes 400 and 500 are shown for determining body characteristics of a subject using the processor 202 and software modules/code instructions stored in memory 204 of FIG. 2, and using the processor 302 and software modules/code instructions stored in memory 304 of FIG. 3.

In the process, the subjects body characteristics are measured via devices 210-224 with processor 202 (FIG. 2) in blocks 402-420. The subjects body characteristics may include, but is not limited to, the user's temperature level, respiratory levels, blood pressure levels, blood oxygen level, IRT, Pulse oximetry (SpO2), heart rate, and EKG.

In block 402, processor 202 detects that the user has pressed the medical module power button to turn the cover on.

In block 404, in response to the power button being turned on, the process connects to an application (also referred to as an App) running on the smartphone 300. The processor 202 may connect to the App via Bluetooth or via any communications channel over, including but not limited to Wi-Fi, cellular or wireline.

In block 406, processor 202 receives a signal from the App in smartphone 300 indicating which test to run. If the indication is to test body temperature, the process executes blocks 408-412. If the indication is an EKG or other test, the process executes block 412-420.

In block 408, the processor 202 reads the initiates the body temperature test. Such initiation may be performed by turning on the temperature sensor.

In block 410, the processor 202 reads the subject/users temperature in response to the user pressing the temperature test button and/or the user placing part of their body next to the temperature sensor.

In block 412, the results of the test are uploaded to the App and the processor 202 then executes block 406 where it receives a signal from the App indicating another test to run.

In block 414, processor 202 runs the EKG test or other tests from one of the other sensors as provided by the App by monitoring the pads and/or thermo sensors for the user/subject to place their fingers on the pads 102 and 104. Such tests may be run periodically or over a predetermined time duration/period of time. The results of such tests may be stored and averaged over the predetermined period of time to determine a typical, normal or average sensor reading for a user.

In block 416, in response to the user placing their fingers in the proper position on the pads 102 and 104, the processor 202 senses the body characteristics as provided by the sensor. If the processor in block 418 determines that the finger was not or improperly placed on the sensor pads 102 and 104, and indication is provided in block 420 to the App that the finger was not properly on the Pads 102 and 104. If the fingers were properly on the pads 102/104 the results of the test are uploaded (via the communications channel) to the smartphone/mobile communications device 100 in block 412. The processor 202 then executes block 406 where it receives a signal from the App indicating another test to run.

Referring to FIG. 5, processor 302 detects in block 502 that the Application has been launched on the smartphone.

In block 504, the processor 302 enables the user to login. The login may require a security feature or incorporate facial recognition to ensure the proper user is running the tests.

In block 506, the process sends a signal to cover (via a communications channel such as Bluetooth to run a test. The order or test that may be run may be preconfigured within the App or may be configured by the user of the smartphone.

In block 508, the processor 302 determines if it has received a signal from the smartphone indicating the fingers were not properly seated on one or more pads 102/104. If the processor 302 has receive such a signal, the processor in block 509 displays or otherwise indications an error message on the display of the smartphone, and then again determines in block 508 if the user's finger is properly seated on the sensors/pads 102 and 104.

If the user fingers were properly seated, in block 510 the processor 302 receives the test results from the cover 100 via one of the communications channels (e.g. via Bluetooth).

In block 512, the processor 302 determines if all tests have been run. If all tests have not been run then in block 506, the App signals the cover 100 via one of the communications channels to run the next test. If all the tests have been run, the App, in block 514, displays the results of one or more of the tests on the display device.

As part of block 514, the results may indicate a specific medical condition, or the App may detect one or more of the following conditions an provide an indication of medical condition needing to be treated further. The conditions detected by the App include:

The infrared temperature of the user combined with the heart rate temperature indicates a specific pathogen and/or physiological health issues in the human body when the detected temperature of the user exceeds 100 degrees Fahrenheit and the Heart Rate of the user exceeds 110% of the average user heart rate detected by the sensor over a predetermined period of time.

The detected temperature of the user detected using the infrared sensor combined with the detected respiratory rate of the user helps detect specific pathogens and/or physiological health issues in the human body. Specifically, an indication of such issues is provided with the users body Temp increases to above 100 F and the detected Respiratory Rate of the user has increased by more than 20% of normal.

The detected breathing rate in combination with the heart rate of the user may be used to detect specific pathogens and/or physiological health issues in the human body. Specifically such an indication may be provided when the detection of the user's Heart Rate shows an increase to more than 10% of normal heart rate. In one implementation, where the normal heart rate (or any sensor rate) is determined by averaging the user's heart rate (or any detected sensor level) over a predetermined prior of time, and when the detected user Respiratory Rate detected by the respiratory sensor has increased by more than 20% of normal respiratory rate.

The infrared temperature feature combined with the breathing rate feature detects specific pathogens and/or physiological health issues in the human body. Specifically when the detected body temperature of the user increases to over 100 degrees F., and the Breathing Rate of the user increase to 20% over the average user breathing rate over a predetermined prior of time a health issue (abnormal heath condition) may be indicated on a display of the communication device.

The blood oxygen rate feature and the breathing rate feature detects specific pathogens and/or physiological health issues in the human body. Specifically upon the users breathing rate increase up by more than 20% of normal and the users SpO2 drops 3% or below 94% oxygen saturation level of the users blood, a health issue may be indicated.

The users infrared temperature feature combined with the detected blood oxygen rate of the user may indicate specific pathogens and/or physiological health issues in the human body. Specifically when the users body temperature increases to greater than 100 F and the users SpO2 level drops 3% or below 94% oxygen saturation, a health issue may be indicated.

The infrared temperature feature combined with the blood oxygen rate feature and the breathing rate feature detects specific pathogens and/or physiological health issues in the human body. Specifically when the detected Temperature of the user increases to above 100 degrees F., and the detected Breathing Rate of the user increases by more than 20% of normal and the detected SpO₂ of the user drops 3% of normal or below 94% oxygen saturation a health issue may be indicated.

Further it has been determined that any combination of detected Temperature, with Heart rate, respiratory rate, oxygen level and/or blood pressure of the user may be used to indicate a health issue or presence of a pathogen in a user.

While the above detailed description has shown, described and identified several novel features of the invention as applied to a preferred embodiment, it will be understood that various omissions, substitutions and changes in the form and details of the described embodiments may be made by those skilled in the art without departing from the spirit of the invention. Accordingly, the scope of the invention should not be limited to the foregoing discussion but should be defined by the appended claims.

What is claimed is:

1. A method for determining body characteristics of a subject using a mobile communications device and a cover of the mobile communications device, the method comprising:
coupling a cover having sensors for determining body characteristics with the mobile communications device to at least partially enclose the mobile communications device;
executing an application on the mobile communications device that signals via a communications channel one or more test criteria to an application executing by a processor in the cover;
engaging sensors embedded in the cover with the subject to detect the subject's body characteristics;
in response to the subject engaging with the sensors and the received one or more test criteria, sensing with the cover one or more body characteristics of the subject;
sending a signal with the processor from the cover to the mobile communications device via the communications channel, the signal indicating the sensed body characteristics; and
displaying one or more tests results on the mobile communications device in response to reception of the signal indicating the sensed body characteristics.

2. The method as recited in claim 1, wherein the sensors include at least one of an infrared thermo Sensor, a Pulse oximetry (SpO₂) sensor, a heart rate sensor, an Eco-Cardiogram (EKG) Sensor, a Blood Pressure Sensor, a temperature sensor, a breathing rate sensor, and a respiratory level sensor.

3. The method as recited in claim 1, further comprising periodically sensing with the cover one or more body characteristics of the subject over a predetermined time duration; and
storing results of sensing with the cover one or more body characteristics of the subject over a predetermined time duration to determine at least one of a typical, normal or average sensor reading for the subject.

4. The method as recited in claim 3, in response to a sensor reading exceeding a predetermined percent of at least one of the typical, normal or average sensor reading for the subject and at least one other sensor exceeding a predetermined percent of at least one of the typical, normal or average sensor reading or another sensor for the subject, indicating a indicating an abnormal health condition with the communications device.

5. A mobile communications device cover comprising:
an outside wall and a bottom panel for engaging with the mobile communications device to at least partially enclose the mobile communications device;
a plurality of sensors coupled with the bottom panel for determining body characteristics; and
a processor coupled with the cover to communicate with the mobile communications device via a communications channel, to receive one or more signals from the mobile communications device to initiate one or more test criteria, to respond to the one or more signals to execute a test application that engages sensors embedded in the cover to sense the subjects body characteristics, and to send a signal with the processor from the cover to the mobile communications device via the communications channel, the signal indicating the sensed body characteristics, such that the one or more tests results are displayed on the mobile communications device in response to reception of the signal indicating the sensed body characteristics.

6. The apparatus as recited in claim 5, wherein the sensors include at least three of an infrared thermo Sensor, a Pulse oximetry (SpO₂) sensor, a heart rate sensor, an Eco-Cardiogram (EKG) Sensor, a Blood Pressure Sensor, a temperature sensor, a breathing rate sensor, and a respiratory level sensor.

7. The apparatus as recited in claim 5, wherein the sensors include an infrared thermo Sensor, a Pulse oximetry (SpO₂) sensor, a heart rate sensor, an Eco-Cardiogram (EKG) Sensor, a Blood Pressure Sensor, and either a breathing rate sensor or a respiratory level sensor.

8. A method of detecting an illness of a user with a mobile communications device cover having a processor coupled with a plurality of sensors, the comprising:
engaging the mobile phone cover with the mobile communications device to attach to and at least partially enclose the mobile communications device;
executing an application on the mobile communications device that signals via a communications channel one or more test criteria to an application executing by a processor in the cover;
engaging sensors embedded in the cover with the subject to detect the subject's body characteristics, the sensors including at least two of an infrared thermo Sensor, a Pulse oximetry (SpO₂) sensor, a heart rate sensor, an Eco-Cardiogram (EKG) Sensor, a Blood Pressure Sensor, a temperature sensor, a breathing rate sensor, and a respiratory level sensor;
in response to the subject engaging with the sensors and the received one or more test criteria, sensing with the cover one or more body characteristics of the subject, the body characteristics including at least one of temperature, heart rate, EKG, pulse oximetry, blood pressure, breathing rate and respiratory level;
sending a signal with the processor from the cover to the mobile communications device via the communications channel, the signal indicating the sensed body characteristics;
determining at least one of a health condition or a pathogen of the subject based on the indicated sensed body characteristics; and
displaying the at least one of the health condition or the pathogen of the subject on the mobile communications device in response to reception of the signal indicating the sensed body characteristics.

9. The method as recited in claim 8, further comprising periodically sensing with the cover one or more body characteristics of the subject over a predetermined time duration; and
storing results of sensing with the cover one or more body characteristics of the subject over a predetermined time duration to determine at least one of a typical, normal or average sensor reading for the subject.

10. The method as recited in claim 9, further comprising in response to a sensor reading exceeding a predetermined percent of at least one of the typical, normal or average sensor reading for the subject and at least one other sensor exceeding a predetermined percent of at least one of the typical, normal or average sensor reading or another sensor for the subject, indicating a indicating an abnormal health condition with the communications device.
